(19) United States
(12) Patent Application Publication
(10) Pub. No.: US 2015/0265397 A1
(43) Pub. Date: Sep. 24, 2015

(54) METHOD FOR TRANSPLANTING A PART OF THE CORNEA AND A SURGICAL MICROSCOPE THEREFOR

(71) Applicant: Carl Zeiss Meditec AG, Jena (DE)

(72) Inventors: Christoph Hauger, Aalen (DE); Joachim Steffen, Westhausen (DE)

(21) Appl. No.: 14/666,129

(22) Filed: Mar. 23, 2015

Related U.S. Application Data
(60) Provisional application No. 61/968,729, filed on Mar. 21, 2014.

(51) Int. Cl. A61F 2/14; A61F 9/008

(52) U.S. Cl. CPC A61F 2/145; A61F 9/008

(57) ABSTRACT

A method for transplanting an endothelial-layer/descemet-layer from a donor organ to a receiver organ include: staining the endothelial-layer/descemet-layer blue in both the donor organ and the receiver organ; making respective incisions of the same diameter into the endothelial-layer/descemet-layer in both the donor organ and the receiver organ using a yellow laser light; preparing the endothelial-layer/descemet-layer from the donor organ and removing the endothelial-layer/descemet-layer within the in particular circular cut in the receiver organ; and, inserting the donor preparation, more particularly circular donor preparation, into the corresponding cutout in the receiver organ. A correspondingly configured surgical microscope is provided.

### CROSS REFERENCE TO RELATED APPLICATION

This application claims priority of U.S. provisional patent application Ser. No. 61/968,729, filed Mar. 21, 2014, the entire content of which is incorporated herein by reference.

### FIELD OF THE INVENTION

The invention relates to a method for transplanting part of the cornea and a correspondingly embodied surgical microscope.

### BACKGROUND OF THE INVENTION

Various surgical processes are known in the field of ophthalmology. By way of example, the method of a complete corneal transplant has been applied for a number of years. To this end, the cornea is removed from a donor organ and transplanted into the eye of a receiver. However, as a result of the massive intervention, an effect occurring here is that it may take up to half a year for the transplanted cornea to once again be completely clear, and so possibly significant visual impairments may occur up until that point.

Furthermore, the so-called DMEK, denoting “Descemet membrane endothelial keratoplasty”, is known. By way of example, this method is applied in the case of patients with endothelial corneal disorders. Only the so-called Descemet membrane with corneal endothelial cells is still transplanted with this method. By way of example, such a method is described in “Der Ophthalmologe 2010, Zeitschrift der Deutschen Ophthalmologischen Gesellschaft” [The Ophthalmologist 2010, Journal of the German Ophthalmological Society], page 369 ff., C. Cursiefen, F. E. Kruse, Springer Medizin. Here, a corneal/scleral disk with endothelial cells on the donor organ is clamped into a Hanna punch under light suction. By way of example, the cornea is stained with trypan blue and the area of the corneal endothelium to be punched is marked under water using a MORIA trepan with a diameter of 8 to 10 mm. Subsequently, the peripheral Descemet membrane is scored in a circular manner using a sharp knife and the delicate structure with a thickness of approximately 15 μm is lifted off via two ligature forceps, for example. In the receiver organ, the diseased layer of the corneal endothelium is, for preparation purposes, mechanically scored in principally the same manner, removed, and replaced by the donor tissue. In order to ensure a precise fit of the donor tissue, it is possible to pump air in such a way that the air bubble presses the donor tissue against the corneal tissue present.

However, it was found to be disadvantageous here that the mechanical scoring or separating out, both in the donor tissue and in the receiver tissue, can lead to deviations, for example, in the diameter of the corneal/scleral disk to be transplanted in relation to the diameter of the tissue removed in the receiver organ. The absolute roundness thereof is not ensured either.

### SUMMARY OF THE INVENTION

Proceeding from the above, it is an object to specify a method for transplanting the Descemet membrane and, possibly, the corneal endothelium, in which a precise fit of the tissue to be transplanted is ensured. Furthermore, the healing process is intended to be accelerated. Finally, an appropriately embodied surgical microscope is to be specified so as to be able to carry out the method.

This object is achieved with the method for transplanting an endothelium-layer/descement-layer from a donor organ to a receiver organ. The method includes the steps of: coloring the endothelium-layer/descement-layer in the donor organ and in the receiver organ; making incisions of equal diameter in the endothelium-layer/descement-layer of the donor organ and of the receiver organ through an application of yellow laser light; preparing the endothelium-layer/descement-layer from the donor organ as a donor specimen; removing endothelium-layer/descement-layer within incision in the receiver organ thus generating a recess therein; and, inserting the donor specimen into the recess in the receiver organ.

### BRIEF DESCRIPTION OF THE DRAWING

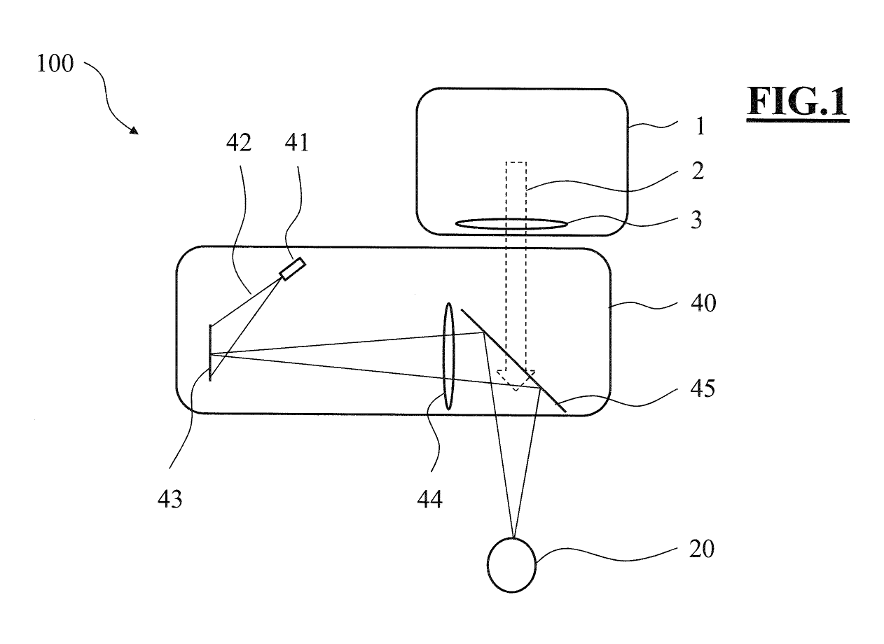

The invention will now first be described in detail and then with reference to the single FIGURE of the drawing (FIG. 1) which shows a schematic illustration of an embodiment of an ophthalmic device with a laser apparatus according to the invention.

### DESCRIPTION OF THE PREFERRED EMBODIMENTS OF THE INVENTION

A basic concept of the invention includes the preparation in the donor tissue and in the receiver tissue no longer being performed by mechanical scoring but rather, according to the invention, the endothelial layer/Descemet membrane layer being stained by trypan blue or any other suitable dye in both the donor organ and receiver organ. Subsequently, these two layers in both the donor organ and the receiver organ are respectively cut by a yellow laser, in particular in a circular shape, for example with a diameter of 8 to 10 mm. To this end, a surgical microscope for anterior chamber surgery on the eye, known per se, can be equipped with, for example, an appropriately embodied laser for the yellow wavelength range. By way of example, this can be a laser which is available under the trade name “Visulas Trion VITE” from the applicant. Here, the surgical microscope or the laser is configured to enable the light of the laser to be guided over a path which, in particular, is circular in order to cut out the corneal/scleral disk with the desired diameter. Preferably, such a laser emits the laser light in pulses with a length of 20 to 50 ms. In principle, the whole ring or circle can also be generated in one go and, for example, be directed onto the tissue for half a second in each case. Here, the beam of the laser, regardless of whether it is guided along the circular path in a scanning manner or whether the circle is formed overall, can have a width of, preferably, 100 μm. In particular, what is achieved by combining trypan blue as a dye with a wavelength in the yellow range from 525 to 675 nm, more particularly from 550 to 610 nm, particularly preferably between 580 and 610 nm, in particular 591 or 599 nm, is that the laser light is only absorbed by the dye within the stained layer or within the stained layers, the dye having its absorption maximum in the specified ranges of the wavelength, such that a cut is brought about through the corneal tissue at the desired depth. In particular, the stroma of the cornea, which has not been stained, is virtually transparent to the yellow laser light, and so no energy is absorbed there and, in particular, there is no tissue damage to the receiver organ. As a result of the dye, the light energy of the laser is absorbed and there is a thermal reaction in the tissue, leading to the cut being formed. It is understood that, to this end, the surgical microscope or a yellow laser integrated therein is embodied in such a way that a preferably circular cut can be performed through the endothelial layer/Descemet membrane layer. To this end, the laser is focused onto this layer or layers. After the endothelial layer/Descemet membrane layer with the desired diameter has been cut out of the donor organ, that is, the donor cornea, this layer/these layers can be removed mechanically in a manner known per se, as is described in the aforementioned publication. In the receiver organ, the endothelial layer/Descemet membrane layer stained blue within the circular cut applied there is scraped off or separated within the circular cut formed therein in a manner known per se. This is brought about on the inner side of the cornea facing the interior of the eye. Subsequently, the preparation obtained from the donor organ is placed onto the remaining corneal stroma from the inside in a manner known per se and, for example, positioned via an air bubble and pressed against the corneal stroma.

Respectively selecting the same diameter for the cut line in the donor organ and in the receiver organ ensures that the donor preparation can be inserted with precise fit into the prepared cutout in the receiver organ. The straight edges generated by the cutline ensure faster and improved healing of the donor layer with the receiver organ, that is, the endothelial layer and, optionally, the Descemet membrane layer.

It is understood that markings can be applied to the donor preparation in a manner known per se so as to ensure the correct location and alignment thereof in the receiver organ. Furthermore, it is clear that the corresponding, in particular circular cuts on the receiver organ during the operation and on the donor organ prior to the operation need not necessarily be carried out with the same surgical microscope or laser. All that is required is that, in the case of circular cuts, these in each case have the same diameter in order to ensure the precise fit.

With respect to the embodiment of the yellow laser, it is suggested that this is tunable at least over a certain wavelength range, more particularly from 525 to 675 nm, more particularly from 550 to 610 nm, particularly preferably between 580 and 610 nm. A wavelength of 591 or 599 nm is particularly preferred so as to obtain ideal fit to the absorbing power of the trypan blue dye in the tissue. It is also clear that the laser is operated at such a power that ensures the in particular circular cut being carried out without, in the process, surrounding tissue, particularly in the receiver organ, being damaged.

Other than the trypan blue dye, it is also possible, in principle, to use different dyes for staining the endothelial layer/Descemet membrane layer. It is understood that when other dyes are used, the corresponding color or wavelength of the laser is tuned to the respective dye. Consequently, this ensures that only the desired layers are in each case correspondingly stained, with the absorbing power of the dye in a specific wavelength range ensuring that only light with the corresponding wavelength is absorbed in the stained layer and, for example, the corneal stroma lying therebelow is not injured by the laser light. Thus, an in particular circular cut is generated at the desired depth.

Furthermore, a protective screen can be placed directly onto the pupil for protecting the retina in the receiver eye. Since the eye needs to be opened mechanically via a scalpel in order to introduce the dye, the protective screen can suppress the ingress of excess light into the eye such that the retina is not damaged.

In principle, instead of a laser which substantially emits a time-constant laser beam with a desired wavelength or which, as described above, is clocked, it is also possible to use a laser which, for example, is clocked in the femtosecond range. Clock frequencies of 100 fs to 500 ms offer themselves in this case. If such a pulsed laser is focused exactly onto the endothelial layer/Descemet membrane layer to be cut, it is possible, in principle, to dispense with the preceding staining of these layers, for example, using trypan blue, since tissue is only ablated by the pulsed laser in the focus thereof, that is, in the endothelial layer/Descemet membrane layer. Here, the tissue itself is disintegrated by the laser pulses without this requiring the energy-absorbing effect of the dye. Consequently, it is likewise possible to generate an in particular circular cut.

Before the, for example, circular cut is carried out, particularly in the receiver organ, appropriate preparation planning can be undertaken with ophthalmic systems known per se. By way of example, the circle with the desired diameter is optically projected onto the receiver eye or the endothelial layer/Descemet membrane layer via the “Callisto Eye” system of Carl Zeiss Meditec AG such that, for example, the surgeon can set the diameter or the size of the region to be ablated in a desired manner.

In principle, it is possible that the material in the receiver organ within the circular cut is not mechanically ablated in a manner known per se, that is, for example, scraped off, but that the endothelial layer/Descemet membrane layer is ablated by the laser itself. To this end, the whole layer is stained, for example by trypan blue, and ablated by the action of the yellow laser light. However, there could be thermal damage to the surrounding tissue in this case and the healing process of the donor tissue with the receiver tissue could be disturbed.

FIG. 1 shows a schematic illustration of an embodiment of an ophthalmic device 100 with a laser apparatus 40 according to the invention in an arrangement below a surgical microscope 1 with a magnification unit 3, which is only depicted schematically. A line of sight 3 of a surgeon through the surgical microscope 1 is depicted by the flash 2. This ophthalmic device is used as described above to transfer the endothelial layer/Descemet membrane layer from a donor organ to a receiver organ. It is obvious that the eye 20 depicted in FIG. 1 could be the donor organ or the receiver organ.

The light 42 emerging from a laser 41 is incident on a device 43 as a contour-shaping beam deflection device. This device 43 is configured to set the laser light 42, especially a yellow laser light, in such a way that the laser light 42 for example in the form of a ring-shaped or oval line contour, is guided to an eye 20 via imaging optics 44 and a coupling element 45.

By this, the ophthalmic device 100 is configured to enable the light of the laser to be guided over a path which, in particular, is circular in order to cut out the corneal/scleral disk of the eye 20 with the desired diameter.

It is understood that the foregoing description is that of the preferred embodiments of the invention and that various changes and modifications may be made thereto without departing from the spirit and scope of the invention as defined in the appended claims.

What is claimed is:

1. A method for transplanting an endothelium-layer/descement-layer from a donor organ to a receiver organ, the method comprising the steps of:
coloring the endothelium-layer/descement-layer in the donor organ and in the receiver organ;
making incisions of equal diameter in the endothelium-layer/descement-layer of the donor organ and of the receiver organ through an application of yellow laser light;
preparing the endothelium-layer/descement-layer from the donor organ as a donor specimen;
removing endothelium-layer/descement-layer within incision in the receiver organ thus generating a recess therein; and,
inserting the donor specimen into the recess in the receiver organ.

2. The method of claim 1, wherein a surgical microscope for anterior chamber surgery having a yellow laser is used to make said incisions.

3. The method of claim 2, wherein the laser operates with a wavelength in a range of 525 nm to 675 nm.

4. The method of claim 2, wherein the laser operates with a wavelength in a range of 550 nm to 610 nm.

5. The method of claim 2, wherein the laser operates with a wavelength in a range of 580 nm to 610 nm.

6. The method of claim 2, wherein the laser operates with a wavelength of 591 nm or 599 nm.

7. The method of claim 1 further comprising the step of protecting the retina of the receiver organ with a protective diaphragm.

8. The method of claim 1, wherein the laser is a pulsed laser.

9. The method of claim 1, wherein the laser is a pulsed laser operating in a femtosecond range.

10. The method of claim 1 further comprising the step of:
projecting a incision line onto the endothelium-layer/descement-layer of the receiver organ with the aid of an optical system of a surgical microscope prior to making the incisions of equal diameter in the endothelium-layer/descement-layer of the donor organ and of the receiver organ, the incision line corresponding to the incision to be made in the endothelium-layer/descement-layer of the receiver organ.

11. The method of claim 1, wherein the incision in the endothelium-layer/descement-layer of the receiver organ defines a receiver organ incision area, the method further comprising the step of:
removing the endothelium-layer/descement-layer of the receiver organ within the receiver organ incision area with the laser.

12. The method of claim 1, wherein trypan blue is used for said coloring the endothelium-layer/descement-layer in the donor organ and in the receiver organ.

13. The method of claim 1, wherein the incisions are circular incisions.

14. A surgical microscope for transplanting an endothelium-layer/descement-layer of a donor organ to a receiver organ, the apparatus comprising:
a yellow laser configured to output yellow light and guide said yellow light over a path in order to make an incision in endothelium-layer/descement-layer of the donor organ and in the endothelium-layer/descement-layer of the receiver organ.

15. The surgical microscope of claim 14, wherein the laser operates with a wavelength in a range of 525 nm to 675 nm.

16. The surgical microscope of claim 14, wherein the laser operates with a wavelength in a range of 550 nm to 610 nm.

17. The surgical microscope of claim 14, wherein the laser operates with a wavelength in a range of 580 nm to 610 nm.

18. The surgical microscope of claim 14, wherein the laser operates with a wavelength of 591 nm or 599 nm.

19. The surgical microscope of claim 14, wherein said path is a circularly-shaped path.

20. The surgical microscope of claim 14, wherein said light generated by said laser generates a circularly-shaped ring.
